(19) United States
(12) Patent Application Publication
(10) Pub. No.: US 2018/0199852 A1
(43) Pub. Date: Jul. 19, 2018

(54) MRI MACHINE AND METHOD FOR MEASURING A HEAD AREA OF A PATIENT

(71) Applicant: DENTSPLY SIRONA Inc., York, PA (US)

(72) Inventors: Erich HELL, Wustweiler (DE); Volker RASCHE, Erbach (DE); Johannes ULRICI, Darmstadt (DE)

(73) Assignee: DENTSPLY SIRONA Inc., York, PA (US)

(21) Appl. No.: 15/868,179

(22) Filed: Jan. 11, 2018

(30) Foreign Application Priority Data
Jan. 13, 2017 (EP) 17151483.9

(51) Int. Cl. A61B 5/055; G01R 33/30; G01R 33/385

(52) U.S. Cl. CPC A61B 5/0555; G01R 33/385; G01R 33/307

(57) ABSTRACT

Described is an MRI machine for measuring a head area of a patient, comprising at least one primary magnetic field unit, at least one gradient coil assembly, a closed patient opening, at least one excitation coil and at least one receiver coil. In a first region, a closed patient opening of the MRI machine comprises a first inner diameter perpendicular to a center axis of the MRI machine and, in the second region, a second inner diameter, which is arranged parallel to the first inner diameter. The first inner diameter is smaller than the second inner diameter, wherein the first region at least partially comprises the head area of the patient, wherein the second region at least partially comprises a torso area of the patient. The MRI machine is arranged obliquely, wherein, in a z-direction relative to a direction of the gravitational force, the center axis of the MRI machine comprises an angle between 20° and 75°.

### TECHNICAL FIELD

This disclosure relates to an MRI machine for measuring a head area of a patient, comprising at least one primary magnetic field unit, at least one gradient coil assembly, a patient opening, at least one excitation coil and at least one receiver coil.

### BACKGROUND

A number of MRI machines for measuring a patient are known from the state of the art.

DE 10 2009 027119 B4 discloses an MRI system for the imaging acquisition of a head area with a permanent magnet, a gradient coil and at least one high-frequency coil, wherein the height of a magnetic field unit can be adjusted on a vertically arranged stand. The magnetic field unit can additionally be pivoted relative to the longitudinal axis of the stand by an angle up to of 45°. The patient assumes a sitting position during the measurement, wherein the head of the patient is positioned relative to the stand by means of a forehead support and earpieces.

With this MRI system, the positioning of the patient relative to the MRI machine is performed in a cumbersome manner in that the patient is moved into a cylindrical patient opening. To do this, a height-adjustable stool and the vertically adjustable stand of the MRI system are adjusted manually in such a way that the head of the patient can be measured.

DE 197 34 138 B2 discloses an MRI machine comprising a gradient coil assembly and an MRI main coil.

US 2013/0023418 A1 discloses an MRI machine with a cooling system consisting of a first stage and a second stage.

The object of the present disclosure is therefore to provide an MRI machine that enables the measurement of a head area of a patient, wherein the dimensions of the MRI machine are as compact as possible and a comfortable positioning of the patient relative to the MRI machine is made possible.

### SUMMARY

Disclosed herein a MRI machine for measuring a head area of a patient, comprising at least one primary magnetic field unit, at least one gradient coil assembly, a closed patient opening, at least one excitation coil and at least one receiver coil, wherein in a first region, a closed patient opening of the MRI machine comprises a first inner diameter perpendicular to a center axis of the MRI machine and, in the second region, a second inner diameter, which is arranged parallel to the first inner diameter, wherein the first inner diameter is smaller than the second inner diameter, wherein the first region at least partially comprises the head area of the patient, wherein the second region at least partially comprises a torso area of the patient, wherein the MRI machine is arranged obliquely, wherein the center axis of the MRI machine comprises an angle between 20° and 75° relative to a direction of the gravitational force.

### BRIEF DESCRIPTION OF THE DRAWINGS

The present disclosure is explained with reference to the drawings. The drawings show:

FIG. 1 an MRI machine for measuring a head area.

### DETAILED DESCRIPTION

The disclosure relates to an MRI machine for measuring a head area of a patient, comprising at least one primary magnetic field unit, at least one gradient coil assembly, a patient opening, at least one excitation coil and at least one receiver coil. In a first region, a closed patient opening of the MRI machine comprises a first inner diameter perpendicular to a center axis of the MRI machine and, in the second region, a second inner diameter, which is arranged parallel to the first inner diameter, wherein the first inner diameter is smaller than the second inner diameter. The first region at least partially comprises the head area of the patient, whereas the second region at least partially comprises a torso area of the patient. The MRI machine is arranged obliquely, wherein the center axis of the MRI machine comprises an angle between 20° and 75° relative to a direction of the gravitational force.

The MRI machine (magnetic resonance imaging machine) is a conventional MRI machine for measuring a head. The MRI machine comprises a measuring volume, wherein the object volume of the object is arranged within the measurement volume in order to measure the object.

In a conventional MRI machine, the measurement is carried out within the object volume by superimposing x, y and z-gradient fields, which are varied temporally, onto the primary magnetic field. The magnetic field in the object can be spatially varied by means of the gradient fields, as a result of which the Larmor frequency of the protons in the object becomes location-dependent to the same degree. A spatial encoding of the excitation of the protons with an excitation pulse generated by the excitation coil and/or a spatial encoding of the measurement signal emitted by the protons and measured by the receiver coil are thus possible.

In a conventional MRI machine, spatial encoding of the voxels of the measuring volume is performed by allocating the selected signals to the individual volume elements (voxels), wherein the spatial encoding is generated with linearly location-dependent magnetic fields (gradient fields). This makes use of the fact that, for one specific particle, the Larmor frequency depends on the magnetic flux density (the greater the field component perpendicular to the direction of the angular momentum of the particle, the higher the Larmor frequency).

One option for using the gradient fields for spatial encoding here is to apply a first gradient during excitation, so that only one slice of the object possesses the proper Larmor frequency, i.e. only the spins of this slice are deflected (slice selection gradient).

A second gradient, transverse to the first, is briefly switched on after excitation and effects a controlled dephasing of the spins in such a way that, in each line of the image, spins with a different phase position process (phase encoding gradient).

During the measurement, the third gradient is switched in a direction that is linearly independent of the two other directions. The third gradient ensures that the spins of each column of the image have a different precession velocity, i.e. transmit a different Larmor frequency (readout gradient, frequency encoding gradient).

Together the three gradients thus bring about an encoding of the signal in three spatial planes. The received signal belongs to a specific slice of the object and contains a combination of frequency and phase encoding, which the computer can convert into a two-dimensional image with a Fourier transformation.

The gradient coil assembly thus consists of a first x-gradient coil, a second y-gradient coil and a third z-gradient coil, wherein the gradient axes of the gradients are arranged linearly independent of one another.

The primary magnetic field unit generates the primary magnetic field and can, for example, consist of at least one permanent magnet, at least one magnetic coil or at least one superconducting magnetic coil.

The patient opening can be shaped as desired and can, for example, comprise a circular shape, an elliptical shape or a rectangular shape. The patient opening must be large enough so that in particular the head area of the patient can fit into the measuring zone of the MRI machine.

The excitation coil and the receiver coil can be separate coils, or they can be combined in one radiofrequency coil.

The center axis of the MRI machine can, for example, coincide with an axis of the substantially cylindrical z-gradient coil. The center axis can also coincide with an axis of symmetry of the patient opening. The center axis of the MRI machine can also coincide with the axis of symmetry of the primary magnetic field of the primary magnetic field unit or it can be oriented parallel to the axis of symmetry of the primary magnetic field.

The first inner diameter of the first region perpendicular to the center axis is disposed parallel to the second inner diameter of the second region. The first inner diameter can be selected to be large enough for the head of the patient to fit inside, for example, whereas the second inner diameter can be selected such that in particular the shoulder area or the torso area of the patient fits inside.

The MRI machine is arranged obliquely, wherein the center axis of the MRI machine comprises an angle between 20° and 75° relative to the direction of the gravitational force. The patient is therefore brought into a sitting position and is moved into the MRI machine at an oblique angle.

One advantage of the present MRI machine is that an obliquely seated patient can be positioned on the patient seat in a comfortable manner and can be moved into the MRI machine fully automatically. This is because the patient can be situated on a patient seat in a reclining, seated position that is comfortable for the patient, and moved into the MRI machine.

A further advantage of the present MRI machine is that the multistage configuration of the patient opening allows the MRI machine to be realized in a more compact design. This is because the shoulder area and the torso area of the patient can be accommodated in the second region with the larger inner diameter. This allows the external dimensions of the MRI machine to be more compact and shorter, without having to restrict the size of the patient opening in the shoulder area.

Another advantage is that the collision of a lower surface of the MRI machine with the thighs or the knees of the patient when the patient is moved into the machine is prevented because, due to the oblique arrangement of the MRI layout, the knees are not bent as much as in an upright position of the patient and, even though the external dimensions of the system are the same, more space is available for thighs and knees due to the larger inner diameter of the patient opening below the head.

The second inner diameter of the second region can advantageously be disposed along a coronally disposed shoulder axis of the torso area of the patient.

The coronal plane is a frontal plane and refers to the plane of motion visible when viewing a person from the front.

The second inner diameter of the second region is therefore selected to be large enough along the shoulder axis of the torso area to also accommodate the torso area of a larger patient.

A first cross section of the patient opening in the first region and/or a second cross section in the second region can advantageously comprise a circular shape, an elliptical shape or a rectangular shape.

The first cross section of the first region and the second cross section of the second region are therefore selected such that the head area of the patient fits into the first region and the torso area of the patient fits into the second region.

In the first region, the patient opening can advantageously comprise the first inner diameter between 250 mm and 650 mm and, in the second region, the second inner diameter between 500 mm and 800 mm.

As a result of the selection of these dimensions for the first inner diameter and the second inner diameter, an average patient can be moved into the patient opening.

The gradient coil assembly can advantageously be arranged within the MRI machine around the patient opening, wherein the gradient coil assembly comprises a first gradient coil inner diameter in the first region of the patient opening and a second gradient coil inner diameter in the second region of the patient opening, which is disposed parallel to the first gradient coil inner diameter, wherein the first gradient coil inner diameter is smaller than the second gradient coil inner diameter.

The gradient coil assembly can therefore consist of an x-gradient coil, a y-gradient coil and a z-gradient coil, wherein the x-gradient coil and the y-gradient coil can be designed as saddle coils, and wherein the z-gradient coil can comprise cylindrically wound coil windings. The gradient coil assembly can thus be mounted directly behind the cladding of the patient opening.

The first gradient coil inner diameter of the first region is thus smaller than the second gradient coil inner diameter of the second region, as is the case for the patient opening, so that the head area of the patient is positioned closer to the first region of the gradient coil assembly and as a result, at the same output of the voltage supply, the gradient fields of the individual gradient coils are stronger.

The gradient coil assembly can advantageously be disposed only in the first region, i.e. in the head area of the patient, so that the second region, i.e. the torso area, is free of the gradient coil assembly.

The gradient coil assembly is therefore disposed only in the head area of the patient, thus allowing the measurement of the patient by means of the MRI machine only in the head area of the patient.

The first region of the patient opening can advantageously comprise a length of at least 150 mm.

As a result, the length of the first region along the center axis of the MRI machine is long enough to accommodate a head area.

The gradient coil assembly advantageously comprises a z-gradient coil for a z-direction, wherein the z-gradient coil comprises coil windings which are wound in a cylindrical manner, wherein the distance between the coil windings varies over the length of the z-gradient coil in the z-direction.

As a result, the winding density of the cylindrically wound coil windings varies along the z-direction of the primary magnetic field.

The winding density of the coil windings of the z-gradient coil can advantageously be configured symmetrically with respect to a plane of the z-gradient coil, which is arranged perpendicular to the center axis of the MRI machine.

The z-gradient field of the z-gradient coil is thus likewise arranged symmetrically with respect to this plane.

The winding density of the coil windings of the z-gradient coil can advantageously be asymmetrical, wherein the coil windings of the z-gradient coil comprise a higher winding density on a side directed toward a torso of the patient compared to the opposite side that is directed toward the head of the patient.

Due to the higher winding density in the torso area, a distance between a lower surface of the gradient coil assembly in the torso area relative to an imaging center can be shorter than the distance of the upper surface of the gradient coil assembly in the head area relative to the imaging center. As a result, the MRI machine can be constructed in a more compact manner.

A transition disposed between the head area of the patient and the torso area of the patient can advantageously be formed between the first region and the second region of the patient opening.

The transition between the first region and the second region can take the form of a step, for example, or even the form of an oblique transition with an angle between 30° and 60° relative to the z-MRI machine.

The MRI machine can advantageously comprise an x-gradient coil and/or a y-gradient coil, which can be designed as saddle coils.

This allows a precise spatial encoding in an x-direction and a y-direction.

The primary magnetic field unit can advantageously comprise at least one superconducting magnetic coil, which is cooled by means of a cooling system that generates the temperatures required for the superconductivity of the magnetic coil.

The superconducting magnetic coil makes a higher primary magnetic field, for example up to 11 tesla, possible.

The cooling system can advantageously comprise a cryostat with helium as the coolant.

An efficient cooling of the cooling system is thereby made possible.

The primary magnetic field unit can advantageously comprise at least one permanent magnet or at least one magnetic coil without cooling.

Therefore, no cooling system is required, as a result of which the MRI machine is less complicated and can thus be produced more cost-effectively.

Advantageously, the patient opening can additionally comprise a third region with a third cross section and a third inner diameter perpendicular to a center axis of the MRI machine, which is disposed parallel to the first inner diameter and wherein the third inner diameter is larger than the second inner diameter of the second region.

The third region can be disposed below the second region and below the first region, so that the patient opening is shaped like a stepped pyramid. In this way, the first region can encompass the head of the patient, the second region the shoulder area of the patient and the third region the abdominal area and the hip area of the patient.

The disclosure further relates to a method for positioning a patient relative to the aforementioned MRI machine, wherein a patient seat is attached to the MRI machine and wherein the patient seat comprises adjusting means. The patient is positioned on the patient seat and is brought into an imaging position by moving the patient at least partially into the MRI machine with the aid of the adjusting means along an axis of travel, until a head of the patient is arranged at least partially in the first region of the patient opening of the MRI machine.

The patient seat is fixedly attached to the MRI machine and by means of the adjusting means allows a precise positioning of the patient, in particular the head of the patient in the first region of the patient opening of the MRI machine.

One advantage of this method is that the patient is moved into the MRI machine with the aid of the adjusting means, and in particular the head of the patient is positioned in a controlled manner in the first region of the MRI machine. Positioning errors of the patient within the MRI machine, which can be caused by faulty manual operation, for example, are thus prevented.

The MRI machine is arranged obliquely, whereby the axis of travel can comprise an angle relative to the center axis of the MRI machine that is disposed within a tolerance angle range between +10° and −10° relative to the center axis of the MRI machine.

This ensures that the patient does not collide with the inner wall of the patient opening when the patient is moved into the MRI machine.

To bring the patient seat into the imaging position, the adjusting means of the patient seat can advantageously be mechanically adjusted by a user.

Therefore, there is no need for an electric motor or an electronic control to adjust the patient seat.

To bring the patient seat into the imaging position, the adjusting means of the patient seat can advantageously be driven by at least one electric motor and controlled by means of a control unit.

The patient seat can thus be adjusted automatically until the head of the patient reaches the first region of the patient opening.

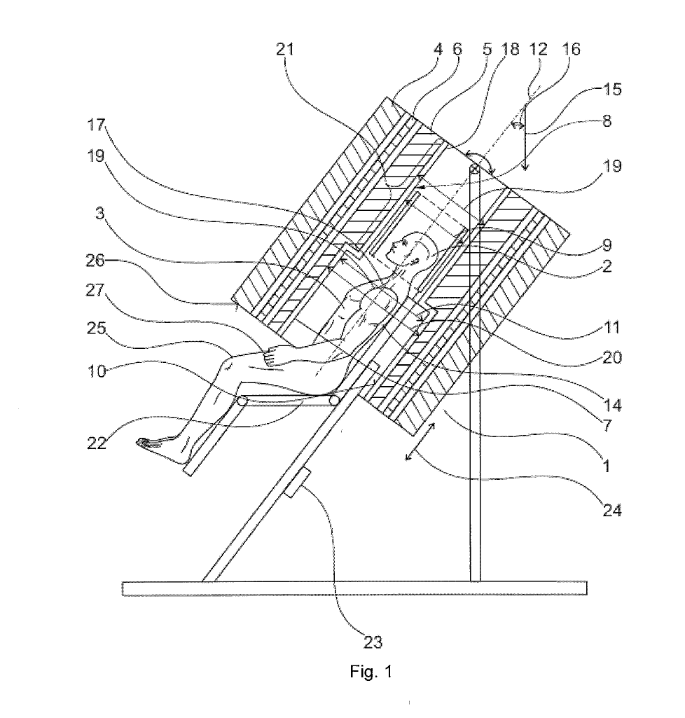

FIG. 1 shows an MRI machine 1 for measuring a head area 2 of a patient 3, comprising at least one primary magnetic field unit 4, at least one gradient coil assembly 5, a high-frequency coil 6, which serves as an excitation coil and as a receiver coil, and a closed patient opening 7. The closed patient opening 7 comprises a first region 8 with a first inner diameter 9 and a second region 10 with a second inner diameter 11. The first inner diameter 9 and the second inner diameter 11 are disposed parallel to one another and perpendicular to a center axis 12, which is represented by a dash-dotted line. At the same time, the center axis 12 is also an axis of symmetry of the two cylindrical regions 8 and 10 of the patient opening 7. In the case shown, the patient 3 is already positioned within the MRI machine in such a way that the head 2 of the patient 3 is located in the first region 8 and a shoulder area 13 and partially a torso area 14 are located in the second region 10 of the patient opening 7. The center axis 12 of the MRI machine 1, which also corresponds to a z-direction of a not depicted z-gradient coil, comprises an angle 16 between 20° and 75° relative to a direction 15 of the gravitational force. In the present case, the first region 8 and the second region 10 of the patient opening 7 are cylindrical. If the second region had an elliptical cross section, the second inner diameter of the second region would be measured along a coronally disposed shoulder axis 17 of the shoulder area 13, which is represented as a cross. The first inner diameter 9 can be between 250 mm and 650 mm, for example, and the second inner diameter 11 can be between 500 mm and 800 mm. The gradient coil assembly 5 in the present case is disposed directly behind a cladding 18 of the patient opening 7, so that the gradient coil assembly 5 in the first region 8 has a smaller gradient coil inner diameter 19 than a second gradient coil inner diameter 20 in the second region 10.

A length 21 of the first region 8 of the patient opening 7 is at least 150 mm. A patient seat 22 is fixedly attached to the MRI machine 1, wherein the patient seat 22 comprises adjusting means 23 that can move the patient seat 22 along an axis of travel 24. The patient 3 is thus positioned on the patient seat 22 and brought into the depicted imaging position, by moving the patient 3 at least partially into the patient opening 7 of the MRI machine 1 with the aid of the adjusting means 23 along the axis of travel 24, until the head 2 of the patient 3 is arranged at least partially in the first region 8. The adjusting means 23 in the present case comprise an electric motor which drives the patient seat, wherein the electric motor is accordingly controlled by means of a control unit to bring the patient seat 22 into the imaging position. In the present case, the orientation of the axis of travel 24 is parallel to the center axis 12 of the MRI machine 1. One advantage of the oblique arrangement of the MRI machine 1 is that the likelihood of a collision is decreased. This is because the patient can be situated in a reclining, seated position on a patient seat and moved into the MRI machine. Therefore, as a result of the oblique arrangement of the MRI layout 1, the knees 25 are not bent as much as in an upright sitting position of the patient, thus preventing the collision of a lower surface 26 of the MRI machine 1 with the thighs 27 of the patient 3 or the knees 25 of the patient 3 when the patient is moved into the machine.

### REFERENCE SIGNS

1 MRI machine
2 Head area
3 Patient
4 Primary magnetic field unit
5 Gradient coil assembly
6 High-frequency coil
7 Patient opening
8 First region
9 First inner diameter
10 Second region
11 Second inner diameter
12 Center axis
13 Shoulder area
14 Torso area
15 Direction
16 Angle
17 Shoulder axis
18 Cladding
19 Gradient coil inner diameter
20 Second gradient coil inner diameter
21 Length
22 Patient seat
23 Adjusting means
24 Axis of travel
25 Knee
26 Lower surface
27 Thigh

1. MRI machine for measuring a head area of a patient, comprising at least one primary magnetic field unit, at least one gradient coil assembly, a closed patient opening, at least one excitation coil and at least one receiver coil,
wherein in a first region, a closed patient opening of the MRI machine comprises a first inner diameter (perpendicular to a center axis of the MRI machine,
wherein in the second region, a second inner diameter is arranged parallel to the first inner diameter,
wherein the first inner diameter is smaller than the second inner diameter,
wherein the first region at least partially comprises the head area of the patient,
wherein the second region at least partially comprises a torso area of the patient,
wherein the MRI machine is arranged obliquely, and
wherein the center axis of the MRI machine comprises an angle between 20° and 75° relative to a direction of the gravitational force.

2. The MRI machine according to claim 1, wherein the second inner diameter of the second region is disposed along a shoulder axis of the torso area of the patient which is arranged in a coronal manner.

3. The MRI machine according to claim 1, wherein a first cross section of the patient opening in the first region and/or a second cross section in the second region comprises a circular shape, an elliptical shape or a rectangular shape.

4. The MRI machine according to claim 1, wherein in the first region, the patient opening comprises the first inner diameter is between 250 mm and 650 mm, and in the second region, the second inner diameter is between 500 mm and 800 mm.

5. The MRI machine according claim 1, wherein the gradient coil assembly is arranged within the MRI machine around the patient opening, wherein the gradient coil assembly comprises a first gradient coil inner diameter in the first region of the patient opening and a second gradient coil inner diameter in the second region of the patient opening, which is disposed parallel to the first gradient coil inner diameter, wherein the first gradient coil inner diameter is smaller than the second gradient coil inner diameter.

6. The MRI machine according to claim 1, wherein the gradient coil assembly is disposed only in the first region and comprises the head area of the patient, and that the second region comprising the torso area is free of the gradient coil assembly.

7. The MRI machine according to claim 1, wherein the first region of the patient opening comprises a length of at least 150 mm.

8. The MRI machine according to claim 1, wherein the gradient coil assembly comprises a z-gradient coil for a z-direction, wherein the z-gradient coil comprises coil windings which are wound in a cylindrical manner, wherein the distance between the coil windings varies over the length of the z-gradient coil in the z-direction.

9. The MRI machine according to claim 8, wherein the winding density of the coil windings of the z-gradient coil is configured symmetrically with respect to a plane of the z-gradient coil, which is arranged perpendicular to the center axis of the MRI machine.

10. The MRI machine according to claim 8, wherein the winding density of the coil windings of the z-gradient coil is asymmetrical, wherein the coil windings of the z-gradient coil comprise a higher winding density on a side directed toward a torso of the patient compared to the opposite side that is directed toward the head of the patient.

11. A Method for positioning a patient relative to an MRI machine according to claim 1, wherein a patient seat is attached to the MRI machine, wherein the patient seat comprises adjusting means, wherein the patient is positioned on the patient seat and is brought into an imaging position by moving the patient at least partially into the MRI machine by means of the adjusting means along an axis of travel, until the head of the patient is arranged at least partially in the first region of the patient opening of the MRI machine.

12. The Method according to claim 11, wherein the adjusting means of the patient seat are mechanically adjusted by a user in order to bring the patient seat into the imaging position.

13. The Method according to claim 11, wherein the adjusting means of the patient seat are driven by at least one electric motor and are controlled by means of a control unit, in order to bring the patient seat into the imaging position.
